(19) United States
(12) Patent Application Publication
(10) Pub. No.: US 2021/0173483 A1
(43) Pub. Date: Jun. 10, 2021

(54) BRAIN ACTIVITY SIGNAL INPUT DEVICE CONTROL

(71) Applicant: HEWLETT-PACKARD DEVELOPMENT COMPANY, L.P., SPRING, TX (US)

(72) Inventor: Vinicius de Nardi Stock da Silva, PORTO ALEGRE (BR)

(21) Appl. No.: 16/762,674

(22) PCT Filed: Feb. 23, 2018
(86) PCT No.: PCT/US2018/019407

§ 371 Date: May 8, 2020

(51) Int. Cl. G06F 3/01; A61B 5/31; A61B 5/369; A61B 5/00

(52) U.S. Cl. CPC G06F 3/015; A61B 5/7475; A61B 5/369; A61B 5/31

(57) ABSTRACT

Example implementations relate to brain activity signal input device control. An example non-transitory machine-readable medium has instructions executable by a processor to interpret a received first brain activity signal associated with a first body movement of four body movements and perform a first action associated with controlling an input device based on the interpreted first signal. Responsive to the first action performance, the instructions can be executable to interpret a received second brain activity signal associated with a second body movement of the four body movements. The instructions can be executable to perform a second action associated with controlling the computing device based on the interpreted second signal.

### BACKGROUND

A brain-computer interface (BCI) is a direct communication pathway between an enhanced or wired brain and an external device. A BCI can be non-invasive such that it is located outside the skull or invasive, such that it is implanted inside the skull during neurosurgery.

### BRIEF DESCRIPTION OF THE DRAWINGS

FIG. 1 illustrates a diagram of a method for brain activity signal input device control according to an example;

FIG. 2 illustrates a device for brain activity signal input device control according to an example;

FIG. 3 illustrates another device for brain activity signal input device control according to an example;

FIG. 4 illustrates yet another device for brain activity signal input device control according to an example;

FIG. 5 illustrates another diagram of a method for brain activity signal input device control according to an example; and

FIG. 6 illustrates yet another diagram of a method for brain activity signal input device control according to an example.

### DETAILED DESCRIPTION

BCIs are based on a response observed in brain activity signals by either executing an actual body movement or imagining the movement of a body part. For instance, imagining the movement of the right hand may cause an event-related desynchronization (ERS) on the left-brain hemisphere, followed by an event-related synchronization (ERS) on the right-brain hemisphere (e.g., the average energy diminishes on the left side and then, sequentially, the energy increases on the right side. These can be identified programmatically and be associated to commands, which can allow for mapping imagination of a movement to a real execution of a specified action. For example, a movement can be imagined without muscles activating. This can be useful, for instance, in users with paralysis, elderly users, etc. Mapping, as used herein, can include associating elements of brain activity signals to a command or body movement.

Some BCI approaches focus on using a cursor to interact with a virtual keyboard. Such approaches try to replicate what a user would normally do with his or her hands. For instance, attempts may be made to give a user a full range of motion of a mouse, which can result in difficulty in control and an increase a quantity of brain activity signals used to perform an action. For instance, an invasive approach may be taken to achieve a signal-to-noise ratio high enough that a system can accurately classify movements. These invasive BCI approaches may result from a surgical operation that may have a permanent effect on a user.

In contrast, some examples of the present disclosure can include interacting with an input device such as a keyboard and/or mouse with reduced stages and brain activity signals as compared to other approaches. For instance, examples of the present disclosure can divide actions into groups, reducing how many brain activity signals are used to complete a desired output. Further, some examples of the present disclosure can be based on a non-invasive BCI such as an electroencephalography (EEG) device (e.g., cap).

For instance, some examples of the present disclosure can include controlling a keyboard and/or mouse to execute actions based on user movement patterns. Through the imagination or execution of movements of a tongue, legs, right hand, left hand, and/or combination thereof, some examples can allow for control of a mobile-like keyboard and a virtual mouse. In some examples, choices associated with control of the keyboard and/or mouse can be performed in a threshold number of stages (e.g., three, four, or five stages). The EEG device, in some examples, can be used for acquisition of brain signals used for classification.

BCIs can be implemented using machine learning for classifying different types of brain activity signals a user executes (e.g., the different movements imagined). Brain activity signals can be processed, and inter-channel interference can be diminished. Because the electrodes in an EEG cap can be affected by all parts of the brain simultaneously, brain activity signals can be isolated from distinct positions of the scalp. For instance, an electrode placed on the right side of the scalp also captures waves being emitted by the left side of the brain. Isolated signals and diminished influence can result in improved mental strategy classification. In some examples, a common spatial pattern (CSP) model or other model can be used during brain activity signal processing.

Responsive to signal processing, features can be extracted to feed a machine learning model (e.g., Naïve Bayes, Linear Discriminant Analysis (LDA), Support Vector Machine (SVM), etc.) that can execute the classification. The model can include features such as root mean square (RMS) values and standard deviations, in some examples. The machine learning model (also known as a classifier) can be fed the model results to determine which mental strategy is applied at a particular time (e.g., the machine learning model can attempt to identify which movement was imagined by the user). In knowledge of the movement that was imagined, a mapping used to associate the brain activity signals to commands that can be executed by a user. An example of a full control flow using EEG signals can include acquiring signals using an electrode cap, which may have Bluetooth modules to transmit. A device receiving the data can include a server processing the EEG signals. Some examples of the present disclosure can include a command interface that can interpret results outputted by the machine learning model. For instance, some examples can control components of a computing device using brain activity signals.

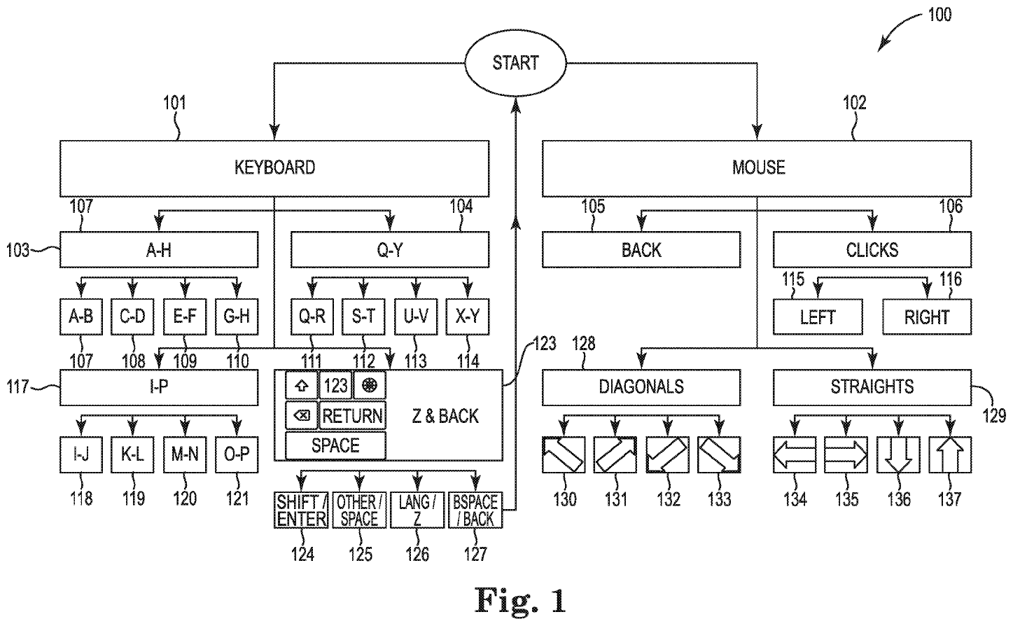

FIG. 1 illustrates a diagram of a method 100 for brain activity signal input device control according to an example. For instance, imaginary movements or actual body movements can be mapped to control a keyboard and/or mouse. The body movements can include, for instance, right-hand movements, left-hand movements, tongue movements, and/or leg movements, among others. In some examples, a combination of body movements can be mapped.

Access to keys of a keyboard and/or actions of a mouse can be divided into groups such that the limited amount of body movements doesn't dictate the variety of keys or actions. For example, by splitting keys or actions into four groups sequentially, a user can get to a desired key or action within three stages. While four groups are described herein, more or fewer movements can be used, resulting in the same number of groups.

Put another way; examples of the present disclosure can include choosing the keyboard or mouse. If the keyboard is chosen, a major key group can be chosen (e.g.; A-H, Q-Y, or miscellaneous). In response; a minor key group can be chosen (e.g., A-B, C-D, E-F, etc.) followed by a desired key (e.g., A, B, C, etc.). Similarly, if the mouse is chosen, a mouse (e.g., a cursor) can be controlled by picking directions to perform movements of a predetermined size (e.g., predetermined number of pixels). Moving the mouse a predetermined distance can result in a better controlled trajectory because command classification errors may produce smaller (e.g., minor) deviations as compared to other approaches.

At 101, the keyboard can be chosen based on a body movement. For instance, a left-hand movement can be mapped to choosing the keyboard. Alternatively, at 102, the mouse can be chosen based on a different body movement. For example, a right-hand movement can be mapped to choosing the mouse.

At 103, responsive to the keyboard being chosen, a body movement, such as a left-hand movement can be mapped to letters A-H, while at 117, a different body movement, such as a right-hand movement, can be mapped to letters I-P. Another body movement such as a leg movement can be mapped to letters Q-Y at 104, and a fourth body movement such as a tongue movement can be mapped to miscellaneous keys at 123.

Responsive to letters A-H being chosen at 103, letters A-B can be chosen at 107 (e.g., by a right-hand movement); letters C-D can be chosen at 108 (e.g., by a left-hand movement), letters E-F can be chosen at 109 (e.g., by a tongue movement), or letters G-H can be chosen at 110 (e.g., by a leg movement), Responsive to letters I-P being chosen at 117, letters I-J can be chosen at 118 (e.g., by a right-hand movement), letters K-L can be chosen at 119 (e.g., by a left-hand movement), letters M-N can be chosen at 120 (e.g., by a leg movement), or letters O-P can be chosen at 121 (e.g., by a tongue movement).

Responsive to letters Q-Y being chosen at 104, letters Q-R can be chosen at 111 (e.g., by a right-hand movement), letters S-T can be chosen at 112 (e.g., by a left-hand movement), letters U-V can be chosen at 113 (e.g., via a tongue movement), or letters X-Y can be chosen at 114 (e.g., via a leg movement). Responsive to miscellaneous keys being chosen at 123, shift/enter keys can be chosen at 124 (e.g., via a right-hand movement), other/space keys can be chosen at 125 (e.g., via a left-hand movement), language/z keys can be chosen at 126 (e.g., via a tongue movement), or backspace/back keys can be chosen at 127 (e.g., via a leg movement).

The keyboard can be exited by choosing the back command, for instance at 123. A user can stay within the keyboard until their desired output is reached. Put another way, mapping of brain activity signals to particular commands can be performed iteratively until the desired output (e.g., a word, a phrase, etc.) is reached. Responsive to the back command being chosen at 123, a user can return to the option of choosing keyboard at 101 or mouse at 102.

Responsive to the mouse being chosen at 102, a back command can be chosen at 105 (e.g., via a right-hand movement), a clicks action can be chosen at 106 (e.g., via a tongue movement), a straights action can be chosen at 129 (e.g., via a leg movement), or a diagonals action can be chosen at 128 (e.g., via a left-hand movement).

Responsive to a clicks action being chosen at 106, a left click action can be chosen at 115 (e.g., via a left-hand movement) or a right click action can be chosen at 116 (e.g., via a right-hand movement). Responsive to a diagonals action being chosen at 128, an upper left diagonal action can be chosen at 130 (e.g., via a left-hand movement), an upper right diagonal action can be chosen at 131 (e.g., via a right-hand movement), a lower left diagonal action can be chosen at 132 (e.g., via a tongue movement), or a lower right diagonal action can be chosen at 133 (e.g., via a leg movement).

Responsive to a straights action being chosen at 129, a left straight action can be chosen at 134 (e.g., via a left-hand movement), a right straight action can be chosen at 135 (e.g., via a right-hand movement), a straight up action can be chosen at 136 (e.g., via a tongue movement), or a straight down action can be chosen at 137 (e.g., via a leg movement). Each movement can move the mouse a pre-determined number of pixels and return to the mouse choice at 102. For instance, a user can stay within the mouse until their desired output is reached. Put another way, mapping of brain activity signals to particular commands can be performed iteratively until the desired output (e.g., a button pressed, etc.) is reached. Responsive to the back command being chosen at 105, a user can return to the option of choosing keyboard at 101 or mouse at 102.

The keyboard groups of FIG. 1 are mapped to commands in the following order: left-hand movement, right-hand movement, tongue movement, and leg movement. The levels are mapped in alphabetical order for the keyboard (e.g., A-H, I-P, Q-Y, and miscellaneous, and a level deeper A-B, C-D, E-F, etc.). However, examples are not so limited. Movements can be mapped to other groups for the keyboard and/or the mouse. The movements can be actual or imaginary, and in some instances, a combination of movements can be used (e.g., tongue and leg movements used in combination to choose a letter).

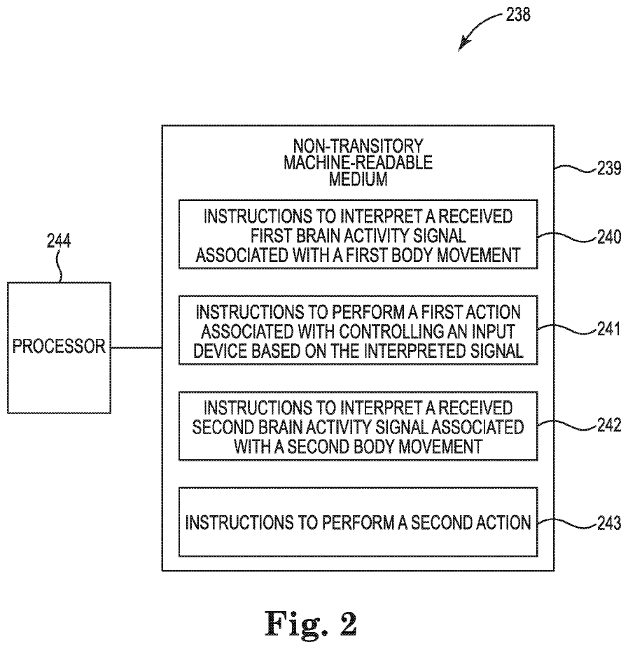

FIG. 2 illustrates a device 238 for brain activity signal input device control according to an example. Device 238 and its components can be akin to devices 345 and 449, as will be discussed further herein. Device 238 can be a computing device in some examples and can include a processor 244. Device 238 can further include a non-transitory MRM 239, on which may be stored instructions, such as instructions 240 241, 242, and 243. Although the following descriptions refer to a processor and a memory resource, the descriptions may also apply to a device with multiple processors and multiple me, and memory resources. In such examples, the instructions may be distributed (e.g., stored) across multiple non-transitory MRMs and the instructions may be distributed (e.g., executed by) across multiple processors.

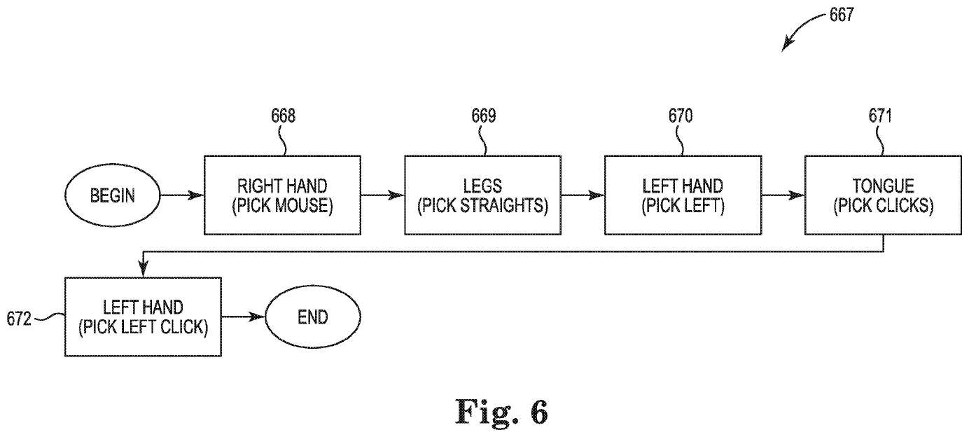

Non-transitory MRM 239 may be electronic, magnetic, optical, or other physical storage device that stores executable instructions. Thus, non-transitory MRM 239 may be, for example, Random Access Memory (RAM), an Electrically-Erasable Programmable Read-Only Memory (EEPROM), a storage drive, an optical disc, and the like on-transitory MRM 239 may be disposed within device 238, as shown in FIG. 6. In this example, the executable instructions 240, 241, 242, and 243 may be “installed” on the device. Additionally and/or alternatively, non-transitory MRM 239 can be a portable, external or remote storage medium, for example, that allows device 238 to download the instructions 240, 241, 242, and 243 from the portable/external/remote storage medium. In this situation; the executable instructions may be part of an “installation package”. As described herein, non-transitory MRM 239 can be encoded with executable instructions for brain activity signal input device control. In some examples, device 238 may use a reduced amount of memory as compared to other approaches. For instance, in some examples, device 238 can use RAM.

Instructions 240, when executed by a processor such as processor 244, can include instructions to interpret a received first brain activity signal associated with a first body movement of four body movements. For example, the first body movement can include a right-hand movement, a left-hand movement, a tongue movement, a leg movement. In some examples, instead of a single body movement, a combination of body movements can be used. The first body movement can be an imagined body movement or an actual body movement. For instance, the first brain activity signal can be interpreted as the first body movement, and the interpretation can include determining with what action to associate the first body movement. The first brain activity signal can be received from a non-invasive EEG device, in some examples.

Instructions 241, when executed by a processor such as processor 244, can include instructions to perform a first action associated with controlling an input device based on the interpreted first signal. The input device, in some examples, can be a keyboard or a mouse. The first action may be chosen from a first set of possible actions such as controlling a keyboard and controlling a mouse. For example, if the interpreted first signal is associated with a tongue movement (or right-hand movement, left-hand movement, leg movement, etc.), which is assigned to controlling a keyboard, control of a keyboard can be performed. Alternatively, if the interpreted first signal is associated with a leg movement (or right-hand movement, left-hand movement, tongue movement, etc.), which is assigned to controlling a mouse, control of a mouse can be performed.

Instructions, 242, when executed by a processor such as processor 244, can include instructions to interpret a received second brain activity signal associated with a second body movement of the four body movements responsive to the first action performance. The second brain activity signal can be received from a non-invasive EEG device, in some examples. The second body movement can be the same or different than the first body movement. For example, the second body movement can include a right-hand movement, a left-hand movement, a tongue movement, a leg movement. The second body movement can be an imagined body movement, and/or an actual body movement. In some examples, a plurality of body movements (e.g., a combination of body movements) can be used associated with performance of an action. For example, a right-hand movement performed at the same time as a left-hand movement (e.g., a combination of body movements) can be associated with an action different than that of just a right-hand movement. In some examples, once keyboard or mouse is chosen (e.g., as the first action performance), further action may be taken to reach a desired output.

Instructions 243, when executed by a processor such as processor 244, can include instructions to perform a second action associated with controlling the computing device based on the interpreted second signal. The second action can include, for instance, selecting groups of keys of a keyboard (e.g., selecting one of a plurality of groups of letter or symbol keys located on the keyboard) and/or selecting a set of mouse movements or mouse action (e.g., selecting one of a plurality of groups of mouse movements or mouse actions). For example, if the first action includes choosing a keyboard, the second action can include choosing a group of keys A-H. If the first action includes choosing a mouse, the second action can include choosing a click action.

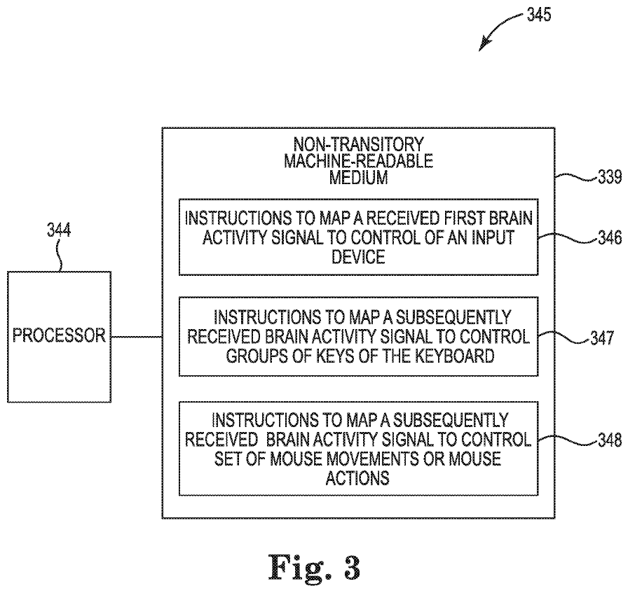

FIG. 3 illustrates another device 345 for brain activity signal input device control according to an example. Device 345 and its components including non-transitory MRM 339 and processor 344 can be akin to devices 238 and 449 and their respective components, as described herein.

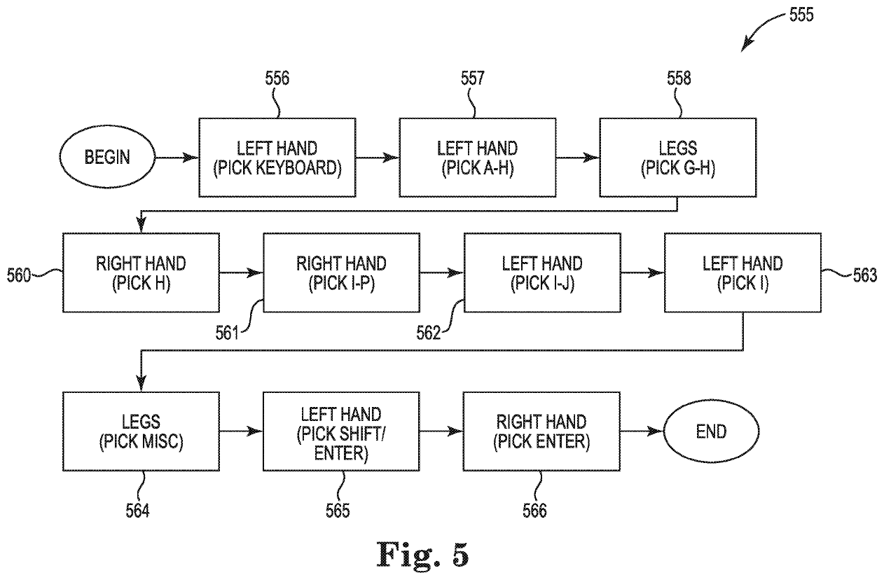

Instructions 346, when executed by a processor such as processor 344, can include instructions to map a received first brain activity signal associated with a first body movement to control of an input device such as a keyboard or mouse. In some examples, the first brain activity signal can be received from a non-invasive EEG device. Instructions 347, when executed by a processor such as processor 344, can include instructions to map subsequently received brain activity signals associated with the four body movements to control a group of keys of the keyboard associated with the subsequently received brain activity signals responsive to the mapping of the received first brain activity signal to control of the keyboard. Example groups of keys include letter keys A through H, letter keys I through P, Letter keys Q through Y, and any remaining keyboard keys, among other possible groupings. The mapping can be performed iteratively until a first desired output is reached. The first desired output, for instance, can be reached in a threshold number (e.g., four) of stages. For example, choosing the letter “h” on the keyboard can be reached in four stages, as will be discussed further herein with respect to FIG. 5.

In some examples, the group of keys of the keyboard associated with the subsequently received brain activity signals can include one of a plurality of different groups of keys on the keyboard. For instance, if keyboard is chosen via the first body movement, a portion of the keyboard (e.g., a group of letters) can be chosen via a subsequent body movement, which can be the same or different than the first body movement (as can be the first brain activity signal and a subsequent brain activity signal).

Instructions 348, when executed by a processor such as processor 344, can include instructions to map subsequently received brain activity signals associated with the four body movements to control a set of mouse movements or mouse actions associated with the subsequently received brain activity signals responsive to the mapping of the received first brain activity signal to control of the mouse. The mapping can be performed iteratively until a second desired output is reached. The second desired output, for instance, can be reached in a threshold number (e.g., four) of stages. For example, choosing to left click with the mouse can be reached in a threshold number of stages, as will be discussed further herein with respect to FIG. 6.

For instance, if the mouse is chosen via the first body movement, an action of the mouse can be chosen via a subsequent body movement, which can be the same or different than the first body movement (as can be the first brain activity signal and a subsequent brain activity signal). The action of the mouse associated with the subsequent brain activity signal and subsequent body movement can be one of a plurality of directional movements and click actions associated with the mouse. For instance, the plurality of directional movements can include movements of a particular distance (e.g., predetermined number of pixels) of a cursor associated with the mouse.

In some examples, up to two additional actions associated with controlling the input device can be performed based on up to two additional subsequently received and interpreted brain activity signals. For instance, if a desired output is not reached subsequent to performance of the second action, additional actions can be performed.

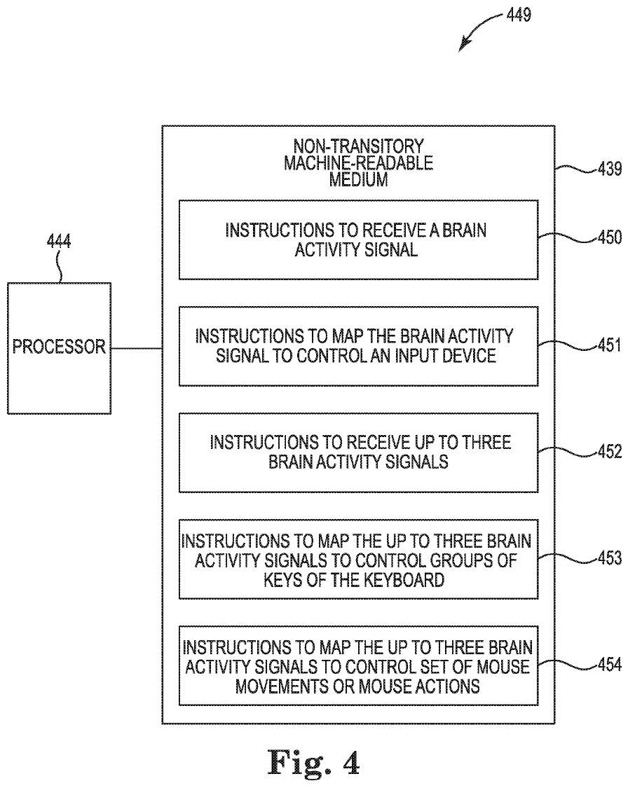

FIG. 4 illustrates another device 449 for brain activity signal input device control according to an example. Device 449 and its components including non-transitory MRM 439 and processor 444 can be akin to devices 238 and 345 and their respective components, as described herein. In some examples, non-transitory MRM 439 comprises RAM.

Instructions 450, when executed by a processor such as processor 444, can include instructions to receive a brain activity signal from a non-invasive EEG device. The brain activity signal can represent one of four body movements. The one of the four body movements can be associated with a particular command in some examples. For instance, the four body movements (and therefore the one of the four body movements) can include a leg, tongue, right-hand, left-hand movement. The particular command can include choosing a keyboard, mouse, group of keys, mouse action, mouse movement, particular key, or mouse action or movement direction, among others. In some examples, the brain activity signal can be received from the non-invasive EEG device subsequent to classification of the brain activity signal. For instance, the received brain activity signal can be fed into a machine learning model before results can be interpreted.

Instructions 451, when executed by a processor such as processor 444, can include instructions to map the brain activity signal to control an input device such as a keyboard or a mouse based on the particular command. For instance, if the brain activity signal is associated with a left-hand movement, which is associated with a keyboard control command, the keyboard can be controlled. Alternatively, the mouse can be controlled if the brain activity signal is associated with a body movement associated with a mouse control command.

Instructions 452, when executed by a processor such as processor 444, can include instructions to subsequently receive up to three brain activity signals representing up to three of the four body movements. The up to three body movements can be associated with up to three particular commands in some examples. For instance, one of the up to three particular commands can include choosing a group of keys if the original particular command is keyboard control or choosing a mouse action if the original particular command is mouse control. The first and up to three subsequent movements can be the same or different, as can the first and up to three subsequent activity signals.

Instructions 453, when executed by a processor such as processor 444, can include instructions to map the up to three subsequently received brain activity signals to control groups of keys of the keyboard based on the up to three particular commands associated with the up to three of the four body movements. The mapping can be done responsive to the mapping of the received brain activity signal to control of the keyboard, for example. Controlling groups of keys of the keyboard can include choosing a group of keys as noted above or choosing a particular key, among others.

Instructions 454, when executed by a processor such as processor 444, can include instructions to map the up to three subsequently received brain activity signals to control a set of mouse movements or mouse actions based on the up to three particular command associated with the up to three body. The mapping can be done responsive to the mapping of the received brain activity signal to control of the mouse, for example. Controlling set of mouse movements or mouse actions can include choosing a mouse action or movement, as noted above or choosing a particular mouse movement direction, among others.

In some examples, the brain activity signal and the up to three brain activity signals can be iteratively mapped until a desired output, such as a word, phrase, or mouse click selection is reached. For instance, if a sentence is desired, a plurality of iterations through the keyboard may be performed before a desired output is reached. Similarly, if a completed form is desired, a plurality of iterations through the keyboard and/or mouse may be performed before a desired output is reached.

FIG. 5 illustrates another diagram 555 of a method for brain activity signal input device control according to an example. The example illustrated in FIG. 5 includes writing the word “HI” in a message application. At 556, keyboard can be chosen using a left-hand movement, and letters A-H can be chosen at 557 using a left-hand movement. Responsive to letters A-H being chosen at 557, letters G-H can be chosen at 558 using a leg movement. A right-hand movement can be used to choose letter H at 560. Because a letter was chosen, a user returns to the beginning of the keyboard options. For instance, at this point, “HI” has been spelled.

At 561, a right-hand movement can be used to choose letters I-P, and letters l-J can be chosen via a left-hand movement at 562. At 563, the letter I can be chosen via a left-hand movement. Because a letter was chosen, the user returns to the beginning of the keyboard options. At 564, miscellaneous keys can be chosen at 564 via a leg movement, and a left-hand movement can be used to choose a shift/enter key at 565. At 566, a right-hand movement can be used to choose an enter key, which can complete the desired output (e.g., send the word, “HI”). The movements described herein are examples. Other movements, whether actual or imagined, can be used for different keys or actions.

FIG. 6 illustrates yet another diagram 667 of a method for brain activity signal input device control according to an example. The example illustrated includes clicking a submit button. For instance, at 668, the mouse can be chosen via a right-hand movement. At 669, a leg movement can be used to choose a straights action, and at 670 a left straight action can be chosen via a left-hand movement. The mouse can be moved a pre-determined number of pixels to the left and return to the mouse level. In this example, the mouse is over the submit button. However, in some example, if the mouse is not over the submit button, further straight left actions can be executed following the same approach. At 671, a click action can be chosen via a tongue movement, and at 672, a left click action can be chosen via a left-hand movement. A left click action can be executed, resulting in the submit button being pressed (e.g., the desired output).

In the foregoing detailed description of the present disclosure, reference is made to the accompanying drawings that form a part hereof, and in which is shown by way of illustration how examples of the disclosure may be practiced. These examples are described in sufficient detail to enable those of ordinary skill in the art to practice the examples of this disclosure, and it is to be understood that other examples may be utilized and that process, electrical, and/or structural changes may be made without departing from the scope of the present disclosure.

The figures herein follow a numbering convention in which the first digit corresponds to the drawing figure number and the remaining digits identify an element or component in the drawing. Elements shown in the various figures herein may be added, exchanged, and/or eliminated so as to provide a number of additional examples of the present disclosure. In addition, the proportion and the relative scale of the elements provided in the figures are intended to illustrate the examples of the present disclosure and should not be taken in a limiting sense. Further, as used herein, “a number of” an element and/or feature may refer to one or more of such elements and/or features.

What is claimed:

1. A non-transitory machine-readable medium comprising instructions executable by a processor of a computing device to:
interpret a received first brain activity signal associated with a first body movement of four body movements;
perform a first action associated with controlling an input device based on the interpreted first signal;
responsive to the first action performance, interpret a received second brain activity signal associated with a second body movement of the four body movements; and
perform a second action associated with controlling the input device based on the interpreted second signal,
wherein the first and the second brain activity signals are received from a non-invasive electroencephalography (EEG) device.

2. The medium of claim 1, wherein the first body movement is an imagined body movement.

3. The medium of claim 1, further comprising instructions executable to perform up to two additional actions associated with controlling the input device based on up to two additional subsequently received and interpreted brain activity signals.

4. The medium of claim 1, wherein the input device is a keyboard or a mouse.

5. The medium of claim 4, wherein the second action comprises selecting a group of keys located on the keyboard.

6. The medium of claim 4, wherein the second action comprises selecting a set of mouse movements or mouse actions.

7. The medium of claim 1, wherein the four movements comprise a right-hand movement, a left-hand movement, a tongue movement, and a leg movement.

8. A non-transitory machine-readable medium comprising instructions executable by a processor of a computing device to:
map a first brain activity signal received from a non-invasive electroencephalography (EEG) device associated with a first of four body movements to control of an input device,
wherein the input device is a keyboard or a mouse;
responsive to the mapping of the received first brain activity signal to control of the keyboard, iteratively map subsequently received brain activity signals associated with the four body movements to control a group of keys of the keyboard associated with the subsequently received brain activity signals until a first desired output is reached,
wherein the first desired output is reached in a threshold number of stages; and
responsive to the mapping of the received first brain activity signal to control of the mouse, iteratively map subsequently received brain activity signals associated with the four body movements to control a set of mouse movements or mouse actions associated with the subsequently received brain activity signals until a second desired output is reached,
wherein the second desired output is reached in the threshold number of stages.

9. The medium of claim 8, wherein the group of keys associated with the second brain activity signal comprises one of:
letter keys A through H;
letter keys I through P;
letter keys Q through Y; and
remaining keyboard keys.

10. The medium of claim 8, wherein the set of mouse movements or mouse actions associated with the second brain activity signal comprises one of a plurality of directional movements and click actions associated with the mouse.

11. The medium of claim 10, wherein the plurality of directional movements comprises movements of a particular distance of a cursor associated with the mouse.

12. A non-transitory machine-readable medium comprising instructions executable by a processor of a computing device to:
receive a brain activity signal from a non-invasive electroencephalography (EEG) device, wherein the brain activity signal represents one of four body movements,
wherein the one of the four body movements is associated with a particular command;
map the brain activity signal to control an input device based on the particular command,
wherein the input device is a keyboard or a mouse;
subsequently receive up to three brain activity signals representing up to three of the four body movements and associated with up to three particular commands;
responsive to the mapping of the received brain activity signal to control of the keyboard, map the up to three subsequently received brain activity signals to control groups of keys of the keyboard based on the up to three particular commands associated with the up to three of the four body movements; and
responsive to the mapping of the received brain activity signal to control a set of mouse movements or mouse actions, map the up to three subsequently received brain activity signals to control a set of mouse movements or mouse actions based on the up to three particular commands associated with the up to three body movements.

13. The medium of claim 12, wherein the four body movements comprise a right-hand movement, a left-hand movement, a tongue movement, and a leg movement.

14. The medium of claim 12, wherein the medium comprises random-access memory (RAM).

15. The medium of claim 12, further comprising instructions executable to iteratively map the brain activity signal and the up to three subsequent brain activity signals until a desired output is reached.
